(19) United States
(12) Patent Application Publication
(10) Pub. No.: US 2007/0188736 A1
(43) Pub. Date: Aug. 16, 2007

(54) Obtaining measurement and baseline signals for evaluating assay test strips

(76) Inventors: Julie E. Fouquet, Portola Valley, CA (US); Bo U. Curry, Redwood City, CA (US)

Correspondence Address: Kathy Manke;Avago Technologies Limited, 4380 Ziegler Road, Fort Collins, CO, 80525, US

(21) Appl. No.: 11/356,159

(22) Filed: Feb. 16, 2006

(51) Int. Cl. G01N 33/48

(52) U.S. Cl. 356/39

(57) ABSTRACT

Systems and methods of obtaining measurement and baseline signals for evaluating assay test strips are described. In one aspect, an assay test strip that has a detection zone with at least one measurement region is held in a respective measurement position for each measurement region in the detection zone. In each measurement position, measurement signals are produced in a measurement data channel in response to light from a respective measurement region of the test strip and baseline signals are produced in a baseline data channel separate from the measurement data channel in response to light from a respective region of the test strip outside of any measurement region. Respective ones of the measurement signals are quantified with respect to corresponding ones of the baseline signals for each measurement position.

### BACKGROUND

Assay test kits are currently available for testing for a wide variety of medical and environmental conditions or compounds, such as a hormone, a metabolite, a toxin, or a pathogen-derived antigen. FIG. 1 shows a typical lateral flow test strip 10 that includes a sample receiving zone 12, a labeling zone 14, a detection zone 15, and an absorbent zone 20 on a common substrate 22. These zones 12-20 typically are made of a material (e.g., chemically-treated nitrocellulose) that allows fluid to flow from the sample receiving zone 12 to the absorbent zone 22 by capillary action. The detection zone 15 includes a test region 16 for detecting the presence of a target analyte in a fluid sample and a control region 18 for indicating the completion of an assay test.

FIGS. 2A and 2B show an assay performed by an exemplary implementation of the test strip 10. A fluid sample 24 (e.g., blood, urine, or saliva) is applied to the sample receiving zone 12. In the example shown in FIGS. 2A and 2B, the fluid sample 24 includes a target analyte 26 (i.e., a molecule or compound that can be assayed by the test strip 10). Capillary action draws the liquid sample 24 downstream into the labeling zone 14, which contains a substance 28 for indirect labeling of the target analyte 26. In the illustrated example, the labeling substance 28 consists of an immunoglobulin 30 with a detectable particle 32 (e.g., a reflective colloidal gold or silver particle). The immunoglobulin 30 specifically binds the target analyte 26 to form a labeled target analyte complex. In some other implementations, the labeling substance 28 is a non-immunoglobulin labeled compound that specifically binds the target analyte 26 to form a labeled target analyte complex.

The labeled target analyte complexes, along with excess quantities of the labeling substance, are carried along the lateral flow path into the test region 16, which contains immobilized compounds 34 that are capable of specifically binding the target analyte 26. In the illustrated example, the immobilized compounds 34 are immunoglobulins that specifically bind the labeled target analyte complexes and thereby retain the labeled target analyte complexes in the test region 16. The presence of the labeled analyte in the sample typically is evidenced by a visually detectable coloring of the test region 16 that appears as a result of the accumulation of the labeling substance in the test region 16.

The control region 18 typically is designed to indicate that an assay has been performed to completion. Compounds 35 in the control region 18 bind and retain the labeling substance 28. The labeling substance 28 typically becomes visible in the control region 18 after a sufficient quantity of the labeling substance 28 has accumulated. When the target analyte 26 is not present in the sample, the test region 16 will not be colored, whereas the control region 18 will be colored to indicate that assay has been performed. The absorbent zone 20 captures excess quantities of the fluid sample 24.

Although visual inspection of lateral flow assay devices of the type described above are able to provide qualitative assay results, such a method of reading these types of devices is unable to provide quantitative assay measurements and therefore is prone to interpretation errors. Automated and semi-automated lateral flow assay readers have been developed in an effort to overcome this deficiency.

In one approach, a reader detects an intensity of a detection signal arising in one or more measurement zones in a detection zone of a lateral flow assay test strip as a result of the presence of an immobilized labeled target analyte complex. The reader generates a baseline of signal intensity from the measurement zones by interpolating between values of the detection signal outside of the measurement zones and inside of the detection zone. In this process, the reader locates a beginning boundary and an ending boundary for the one or more measurement zones on the test strip, allowing an automatic or semi-automatic analytical instrument, or a human reader, to determine certain results of the lateral flow assay. The signals from the measurement zones are quantified or compared with respect to the baseline. Quantified values corresponding to the respective concentration of compounds in different measurement zones may then be compared with one another to detect the presence of antigens in the sample.

What is needed is a diagnostic test system that is capable of respectively obtaining measurement signals and baseline signals from measurement and baseline regions of an assay test strip without requiring complex and resource-intensive analyses of the signal data.

### SUMMARY

In one aspect, the invention features a diagnostic test system for assaying a test strip. The assay test strip has a flow path for a fluid sample, a sample receiving zone coupled to the flow path, and a detection zone that is coupled to the flow path and has at least one measurement region. The diagnostic test system includes a detection system, an alignment system, and a data analyzer. The detection system includes a measurement region detector and a baseline region detector each of which produces signals in response to light from the test strip. The alignment system is configured cooperatively with the detection system to hold the test strip in a respective measurement position relative to the detection system for each measurement region in the detection zone. In each measurement position, the measurement region detector produces measurement signals in a measurement data channel in response to light from a respective measurement region of the test strip and the baseline region detector produces baseline signals in a baseline data channel separate from the measurement data channel in response to light from a respective region of the test strip outside of any measurement region. The data analyzer quantifies respective ones of the measurement signals with respect to corresponding ones of the baseline signals for each measurement position of the test strip relative to the detection system.

In another aspect, the invention features a diagnostic test method for assaying a test strip. In accordance with this inventive method, the test strip is held in a respective measurement position for each measurement region in the detection zone. In each measurement position, measurement signals are produced in a measurement data channel in response to light from a respective measurement region of the test strip and baseline signals are produced in a baseline data channel separate from the measurement data channel in response to light from a respective region of the test strip outside of any measurement region. Respective ones of the measurement signals are quantified with respect to corresponding ones of the baseline signals for each measurement position.

Other features and advantages of the invention will become apparent from the following description, including the drawings and the claims.

### DESCRIPTION OF DRAWINGS

FIG. 1 is a diagrammatic view of a prior art implementation of an assay test strip.

FIG. 2A is a diagrammatic view of a fluid sample being applied to a sample receiving zone of the assay test strip shown in FIG. 1.

FIG. 2B is a diagrammatic view of the assay test strip shown in FIG. 2A after the fluid sample has flowed across the test strip to an absorption zone.

FIG. 3 is a block diagram of an embodiment of a test strip that is loaded into an embodiment of a diagnostic test system.

FIG. 4 is a diagrammatic side view of an implementation of the diagnostic test system shown in FIG. 3 that includes a detection system that is obtaining light intensity measurements from a pair of measurement and baseline regions of a test strip.

FIG. 5 is a diagrammatic side view of the detection system shown in FIG. 4 that is obtaining light intensity measurements from a different pair of measurement and baseline regions of the test strip.

FIG. 6A is a diagrammatic top view of the assay test strip shown in FIG. 3 on an embodiment of a movable support of an alignment system.

FIG. 6B is a sectional view of the test strip and the support shown in FIG. 6A taken along the line 6B-6B.

FIG. 7A is a diagrammatic sectional view of an embodiment of a detent incorporated in an embodiment of the alignment system shown in FIG. 4.

FIG. 7B is a diagrammatic section view of the detent shown in FIG. 7A engaging a first dimple in a rail.

FIG. 7C is a diagrammatic section view of the detent shown in FIG. 7B engaging a second dimple in the rail.

FIG. 8 is a diagrammatic view of an embodiment of the test strip shown in FIG. 5.

FIG. 9 is a diagrammatic view of an embodiment of the test strip shown in FIG. 5.

FIG. 10 is a diagrammatic view of an embodiment of the test strip shown in FIG. 5.

FIG. 11A is a diagrammatic view of an embodiment of the test strip shown in FIG. 5 and an embodiment of a detection system on a portion of the test strip.

FIG. 11B is a diagrammatic view of the detection system shown in FIG. 11A on a different portion of the test strip.

FIG. 12 is a diagrammatic view of an embodiment of the test strip shown in FIGS. 11A and 11B and an embodiment of a detection system on a portion of the test strip.

### DETAILED DESCRIPTION

In the following description, like reference numbers are used to identify like elements. Furthermore, the drawings are intended to illustrate major features of exemplary embodiments in a diagrammatic manner. The drawings are not intended to depict every feature of actual embodiments nor relative dimensions of the depicted elements, and are not drawn to scale.

I. Introduction

The embodiments that are described in detail below are capable of respectively obtaining measurement signals and baseline signals from measurement and baseline regions of an assay test strip without requiring complex and resource intensive analyses of the signal data. In these embodiments, measurement signals are produced in a measurement data channel in response to receipt of light from measurement regions of the test strip and baseline signals are produced in a baseline data channel separate from the measurement data channel in response to receipt of light from respective regions of the test strip outside of any measurement region. Producing the measurement and baseline signals in separate data channels allows these embodiments to evaluate assay test strips with high accuracy and precision while using relatively inexpensive detectors and processing components.

The terms “assay test strip” and “lateral flow assay test strip” encompass both competitive types of assay test strips in which an increase in the concentration of the analyte in the sample results in an increase in the concentration of labels in the test region and non-competitive types of assay test strips in which an increase in the concentration of the analyte in the fluid sample results in a decrease in the concentration of labels in the test region. A lateral flow assay test strip generally includes a sample receiving zone and a detection zone, and may or may not have a labeling zone. In some implementations, a lateral flow assay test strip includes a sample receiving zone that is located vertically above a labeling zone, and additionally includes a detection zone that is located laterally downstream of the labeling zone.

The term “analyte” refers to a substance that can be assayed by the test strip. Examples of different types of analytes include organic compounds (e.g., proteins and amino acids), hormones, metabolites, antibodies, pathogen-derived antigens, drugs, toxins, and microorganisms (e.g., bacteria and viruses).

As used herein the term “label” refers to a substance that has specific binding affinity for an analyte and a detectable characteristic feature that can be is distinguished from other elements of the test strip. The label may include a combination of a labeling substance (e.g., a fluorescent particle, such as a quantum dot) that provides the detectable characteristic feature and a probe substance (e.g., an immunoglobulin) that provides the specific binding affinity for the analyte. In some implementations, the labels have distinctive optical properties, such as luminescence (e.g., fluorescence) or reflective properties, which allow regions of the test strip containing different labels to be distinguished from one another.

The term “reagent” refers to a substance that reacts chemically or biologically with a target substance, such as a label or an analyte.

The term “capture region” refers to a region on a test strip that includes one or more immobilized reagents.

The term “test region” refers to a capture region containing an immobilized reagent with a specific binding affinity for an analyte.

The term “control region” refers to a capture region containing an immobilized reagent with a specific binding affinity for a label.

The term “measurement region” refers to any region of interest on an assay test strip, such as a capture region or a calibration region, that may be measured for the purpose of evaluating the assay test strip. The term “baseline region” refers to any region of an assay test strip outside of a measurement region.

The term “measurement signal” refers to a signal that is produced by an optical detector in response to light received from a measurement region of an assay test strip. The term “baseline signal” refers to a signal that is produced by an optical detector in response to light received from a baseline region of an assay test strip.

The phrase “quantifying a first value with respect to a second value” refers to a process of deriving a final quantified value from a function that compares the first and second values or values respectively derived from the first and second values. The comparison function may include a ratio between the first and second values, a difference between the first and second values, or some other mathematical function of the first and second values.

II. Diagnostic Test System Architecture

A. Overview

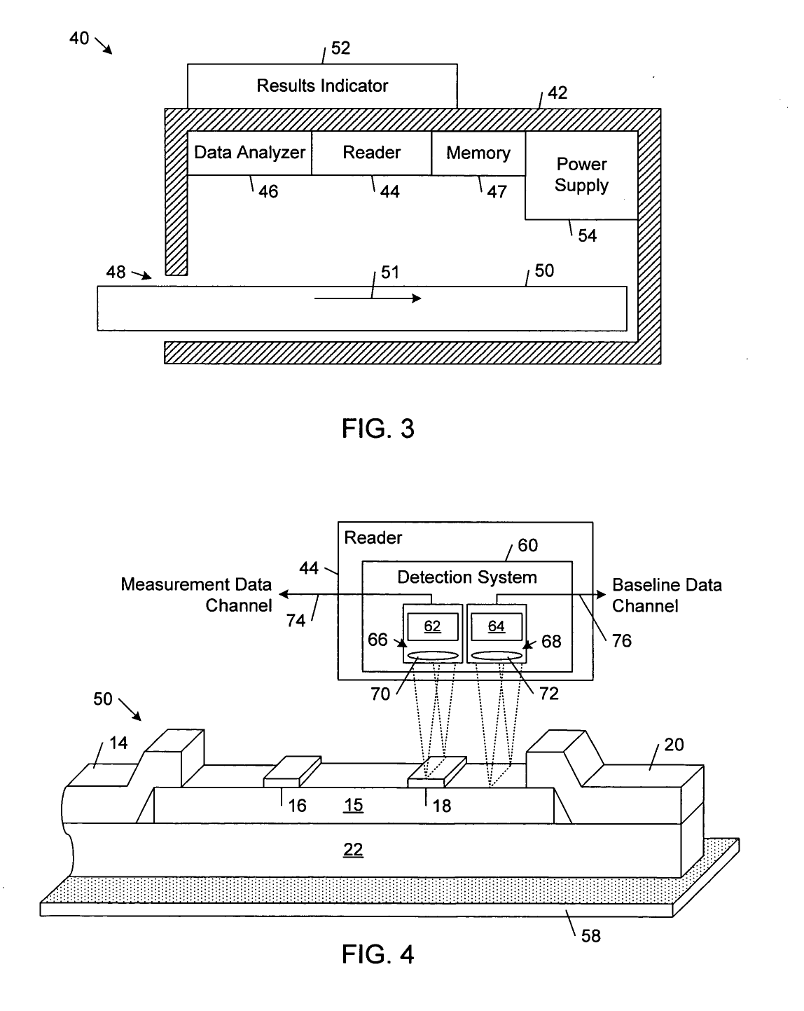

FIG. 3 shows an embodiment of a diagnostic test system 40 that includes a housing 42, a reader 44, a data analyzer 46, and a memory 47. The housing 42 includes a port 48 for receiving a test strip 50. When the test strip 50 is loaded in the port 48, the reader 44 obtains light intensity measurements from the test strip 50. In general, the light intensity measurements may be unfiltered or they may be filtered in terms of at least one of wavelength and polarization. The data analyzer 46 computes at least one parameter from one or more of the light intensity measurements. A results indicator 52 provides an indication of one or more of the results of an assay of the test strip 50. In some implementations, the diagnostic test system 40 is fabricated from relatively inexpensive components enabling it to be used for disposable or single-use applications.

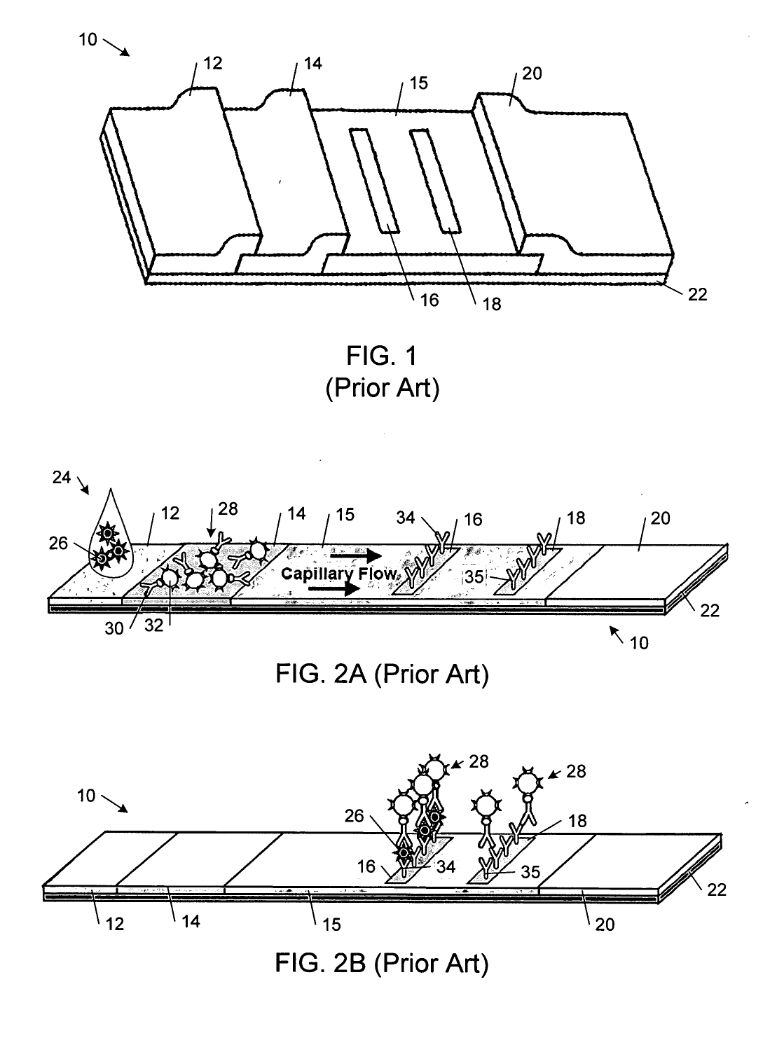

The housing 42 may be made of any one of a wide variety of materials, including plastic and metal. The housing 42 forms a protective enclosure for the reader 44, the data analyzer 46, the power supply 54, and other components of the diagnostic test system 40. The housing 42 also includes an alignment system that mechanically registers the test strip 50 with respect to the reader 44. The alignment system may be designed to receive any one of a wide variety of different types of test strips 50, including test strips of the type shown in FIG. 1.

In the illustrated embodiments, each of the test strips 50 is a non-competitive type of assay test strip that supports lateral flow of a fluid sample along a lateral flow direction 51. Each of the test strips 50 includes a labeling zone containing a labeling substance that binds a label to a target analyte and a detection zone that has at least one test region containing an immobilized substance that binds the target analyte. One or more areas of the detection zone, including at least a portion of the test region, are exposed for optical inspection by the reader 44. The exposed areas of the detection zone may or may not be covered by an optically transparent window.

In other embodiments, the test strips 50 are competitive type of lateral flow assay test strips in which the concentration of the label in the test region decreases with increasing concentration of the target analyte in the fluid sample. Some of these embodiments include a labeling zone, whereas others of these implementations do not include a labeling zone.

Some of these competitive lateral flow assay test strip embodiments include a labeling zone that contains a label that specifically binds target analytes in the fluid sample, and a test region that contains immobilized target analytes as opposed to immobilized test reagents (e.g., antibodies) that specifically bind any non-bound labels in the fluid sample. In operation, the test region will be labeled when there is no analyte present in the fluid sample. However, if target analytes are present in the fluid sample, the fluid sample analytes saturate the label's binding sites in the labeling zone, well before the label flows to the test region. Consequently, when the label flows through the test region, there are no binding sites remaining on the label, so the label passes by and the test region remains unlabeled.

In other competitive lateral flow assay test strip embodiments, the labeling zone contains only pre-labeled analytes (e.g., gold adhered to analyte) and the test region contains immobilized test reagents with an affinity for the analyte. In these embodiments, if the fluid sample contains unlabeled analyte in a concentration that is large compared to the concentration of the pre-labeled analyte in the labeling zone, then label concentration in the test region will appear proportionately reduced.

The reader 44 includes one or more optoelectronic components for optically inspecting the exposed areas of the detection zone of the test strip 50. In some implementations, the reader 44 includes at least one light source and at least one light detector. In some implementations, the light source may include a semiconductor light-emitting diode and the light detector may include a semiconductor photodiode. Depending on the nature of the label that is used by the test strip 50, the light source may be designed to emit light within a particular wavelength range or light with a particular polarization. For example, if the label is a fluorescent label, such as a quantum dot, the light source may be designed to illuminate the exposed areas of the detection zone of the test strip 50 with light in a wavelength range that induces fluorescent emission from the label. Similarly, the light detector may be designed to selectively capture light from the exposed areas of the detection zone. For example, if the label is a fluorescent label, the light detector may be designed to selectively capture light within the wavelength range of the fluorescent light emitted by the label or with light of a particular polarization. On the other hand, if the label is a reflective-type label, the light detector may be designed to selectively capture light within the wavelength range of the light emitted by the light source. To these ends, the light detector may include one or more optical filters that define the wavelength ranges or polarizations axes of the captured light.

The data analyzer 46 processes the light intensity measurements that are obtained by the reader 44. In general, the data analyzer 46 may be implemented in any computing or processing environment, including in digital electronic circuitry or in computer hardware, firmware, or software. In some embodiments, the data analyzer 46 includes a processor (e.g., a microcontroller, a microprocessor, or ASIC) and an analog-to-digital converter. In the illustrated embodiment, the data analyzer 46 is incorporated within the housing 42 of the diagnostic test system 40. In other embodiments, the data analyzer 46 is located in a separate device, such as a computer, that may communicate with the diagnostic test system 40 over a wired or wireless connection.

In general, the results indicator 52 may include any one of a wide variety of different mechanisms for indicating one or more results of an assay test. In some implementations, the results indicator 52 includes one or more lights (e.g., light-emitting diodes) that are activated to indicate, for example, a positive test result and the completion of the assay test (i.e., when sufficient quantity of labeling substance 28 has accumulated in the control region). In other implementations, the results indicator 52 includes an alphanumeric display (e.g., a two or three character light-emitting diode array) for presenting assay test results.

A power supply 54 supplies power to the active components of the diagnostic test system 40, including the reader 44, the data analyzer 46, and the results indicator 52. The power supply 54 may be implemented by, for example, a replaceable battery or a rechargeable battery. In other embodiments, the diagnostic test system may be powered by an external host device (e.g., a computer connected by a USB cable).

III. Obtaining Measurement and Baseline Signals for Evaluating Assay Test Strips

A. Exemplary Multiple Data Channel Architecture for Obtaining Measurement and Baseline Signals

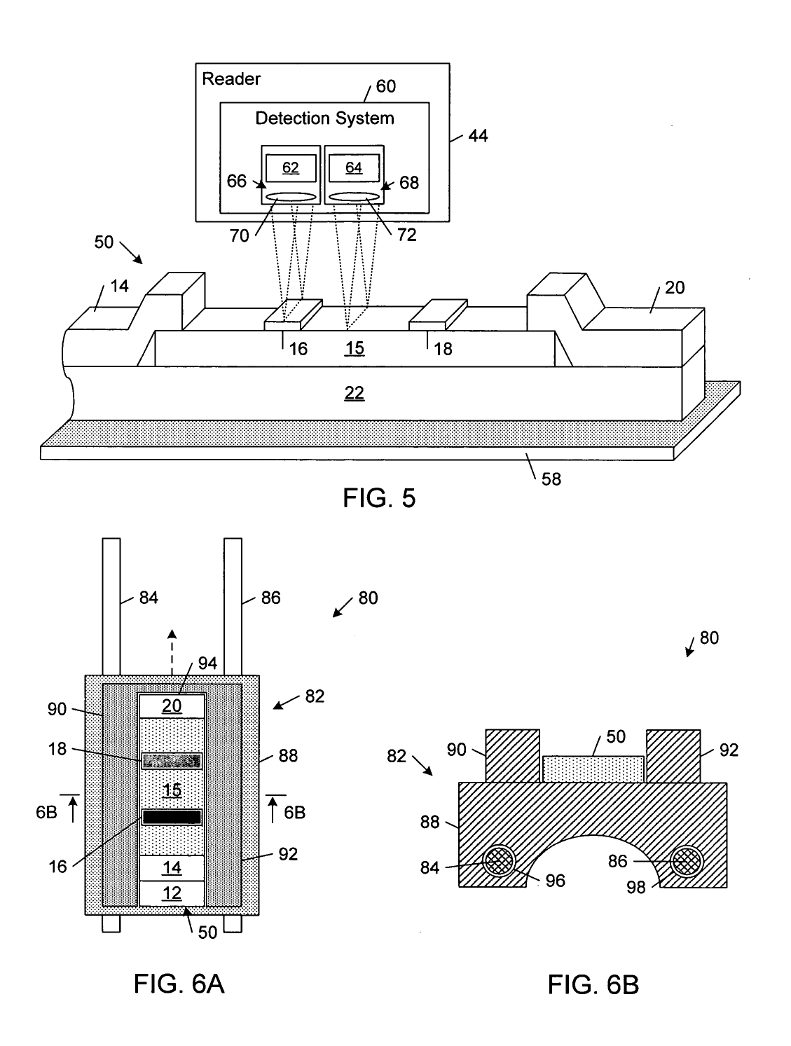

FIGS. 4 and 5 show a portion of an exemplary embodiment of the diagnostic test system 40 in which the assay test strip 50 is held on a support 58 and the reader 44 includes a detection system 60 that produces measurement and baseline signals in response to light received from respective measurement and baseline regions of the test strip 50.

The detection system 60 includes a measurement region detector 62 and a baseline region detector 64, each of which produces signals in respective data channels in response to receipt of light from respective regions of the test strip 50. Each of the measurement region detector 62 and the baseline region detector 64 may be implemented by any type of photodetector device, including a single channel optical detector (e.g., a photodiode device), a linear array optical detector, and a two-dimensional optical detector (e.g., a CCD or CMOS image sensor device). The detection system 60 also may include optical channels 66, 68 that guide light from the test strip 50 onto the respective active areas of the detectors 62, 64. Each of the optical channels 66, 68 may include one or more optical elements 70, 72 (e.g., refractive lenses, diffractive lenses, and optical filters) that intercept and modify the light received from respective regions of the test strip 50.

In some embodiments, the support 58 is configured to move the test strip 50 relative to the detection system 60. In other embodiments, the detection system 60 is configured to move relative to the test strip 50. In some embodiments, both the support 58 and the detection system 60 move relative to each other. Some embodiments of the diagnostic test system 40 include at least one motor that moves the movable ones of the support 58 and the detection system 60. In other embodiments, the movable ones of the support 58 and the detection system 60 are moved manually by a user of the diagnostic test system 40.

As explained in detail below, an alignment system (not shown in FIG. 4) is configured cooperatively with the detection system 60 to guide the movement of at least one of the support 58 and the detection system 60 into a respective measurement position relative to the detection system 60 for each measurement region in the detection zone 15. In each measurement position, the measurement region detector 62 receives light predominantly from a respective one of the measurement regions and the baseline region detector 64 receives light predominantly from a respective baseline region outside of any measurement region. As a result, the measurement region detector 62 produces measurement signals in a measurement data channel 74 in response to receipt of light from a respective measurement region of the test strip 50, and the baseline region detector 64 produces baseline signals in a baseline data channel 76 in response to receipt of light from a respective baseline region of the test strip 50 outside of any measurement region.

FIG. 4 shows the test strip 50 and the detection system 60 in a first measurement position in which the measurement region detector 62 is positioned directly over the control region 18 and the baseline region detector 64 is positioned directly over a baseline region of the detection zone 15 that is adjacent to the control region 18 but outside of any measurement region (i.e., the test region 16 and the control region 18). FIG. 5 shows the test strip 50 and the detection system 60 in a second measurement position in which the measurement region detector 62 is positioned directly over the test region 16 and the baseline region detector 64 is positioned directly over a baseline region of the detection zone 15 that is adjacent to the test region 16 but outside of any measurement region (i.e., the test region 16 and the control region 18).

The data analyzer 46 quantifies the respective ones of the measurement signals in the measurement data channel 74 with respect to respective ones of the baseline signals in the baseline data channel 76 for each measurement position of the test strip 50 relative to the detection system 60. In this process, the data analyzer 46 derives a final quantified value from a function that compares the measurement region values and baseline region values. The measurement region value is derived from the measurement signals (e.g., an average or a peak signal value) and optionally may be calibrated with respect to a dark value that is measured when the light source is turned off. Similarly, the baseline region value is derived from the baseline signals (e.g., an average or a peak signal value) and optionally may be calibrated with respect to a dark value that is measured when the light source is turned off.

The comparison function may include a ratio between the first and second values, a difference between the measurement region value and baseline region value, or some other mathematical function of these values. For example, in some embodiments, the data analyzer 46 quantifies the measurement value in terms of the baseline value to determine a measure of the reflection density of a respective one of the measurement regions of the test strip 50. The reflection density is the logarithm of the reflectance to the base 10, where the reflectance is the ratio of the measurement region value to the baseline region value. The data analyzer 46 may use the reflection density value as an index into a lookup table that maps reflection density values to analyte concentration values.

B. Exemplary Systems for Aligning the Detection System with an Assay Test Strip

As explained in detail below, the test strip 50 and the detection system 60 may be mutually aligned based on the automatic or manual detection of one or more alignment features.

In some embodiments, the alignment system includes one or more alignment features that indirectly indicate the locations of the measurement regions on the test strip 50 based on the relationship between the measurement regions and the support system when the test strip 50 is loaded properly on the support 58. In other embodiments, the test strip 50 includes one or more alignment features that provide a direct indication of the locations of the measurement regions on the test strip 50.

In some embodiments, the alignment system produces a perceptible response (e.g., a clicking sound or resisting manual movement) when the test strip 50 and the detection system 60 are in a measurement position. In other embodiments, the detection system 60 detects when the test strip 50 and the detection system 60 are in a respective measurement position based on the alignment features and generates a control signal that stops the automatic movement of the movable ones of the support 58 and the detection system 60 or triggers a visual or audible indication that the test strip 50 and the detection system 60 are in a measurement position. In other embodiments, the support 58 includes electrically conductive leads that complete a detection circuit in the reader 44 when the test strip 50 is in a measurement position with respect to the detection system 60.

1. Mechanical Position Markers

FIGS. 6A and 6B show top and sectional views of an embodiment of an alignment system 80 that includes a support 82 that holds the assay test strip 50 and slides on a pair of rails 84, 86. The support 82 and the rails 84, 86 may be formed of any one of a wide variety of different materials, including plastic and metal.

The support 82 includes a base 88, a pair of sidewalls 90, 92, and a backstop 94. The sidewalls 90, 92 and the backstop 94 form a receptacle for holding the assay test strip 50. The base 88 includes a pair of bores 96, 98 through which the rails 84, 86 respectively extend. In the illustrated embodiment, the detection system 60 and the rails 84, 86 are fixed to the housing 42 of the diagnostic test system 40 so that when the support 82 slides on the rails 84, 86 the test strip 50 passes directly beneath the measurement region detector 62 and the baseline region detector 64.

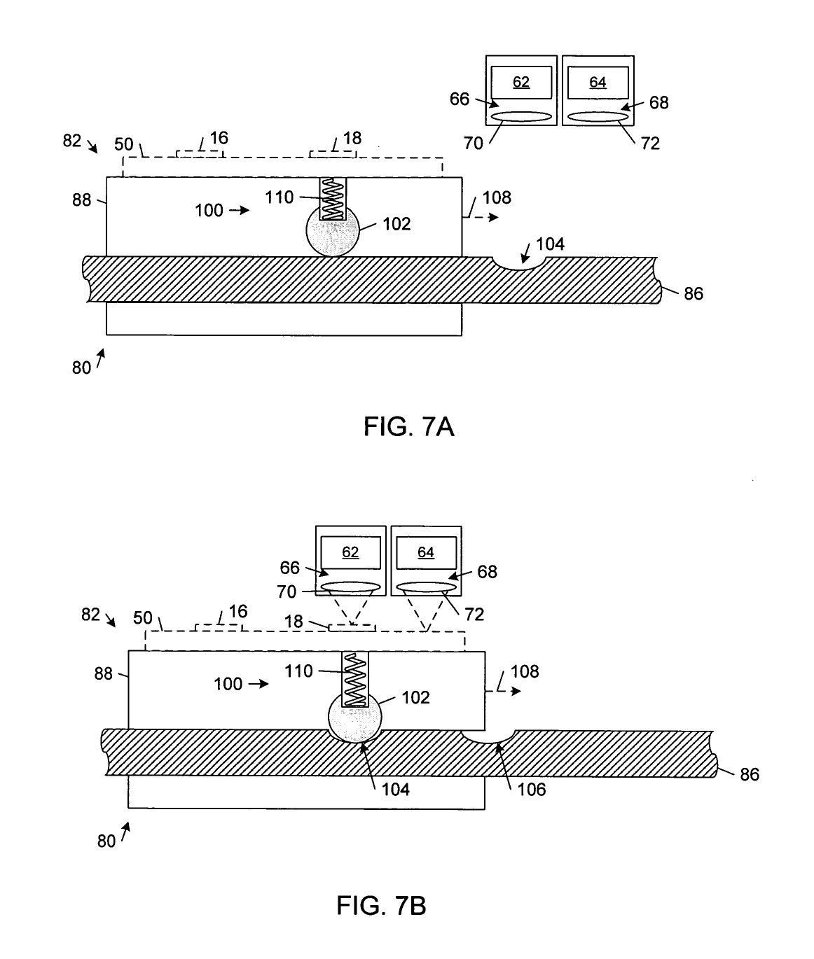

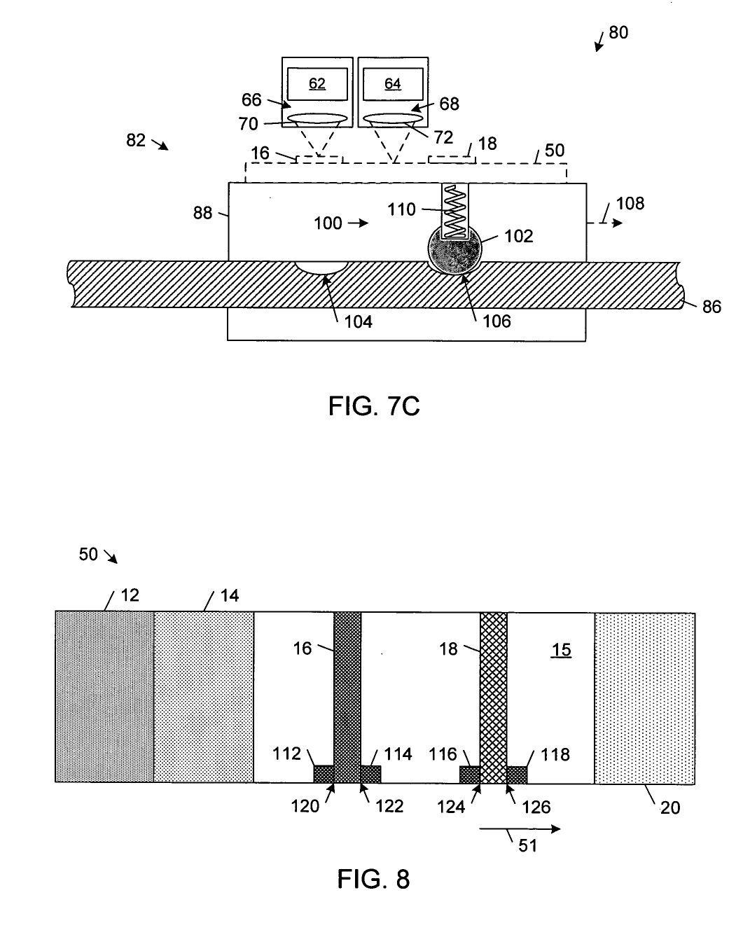

FIGS. 7A, 7B, and 7C show an embodiment of a detent 100 that is incorporated in the base 88 of the support 82. In the illustrated embodiment, the detent 100 is implemented by a spring-loaded ball 102. In other embodiments, the detent 100 may be implemented by a different mechanism (e.g., a catch or dog) for positioning and holding the base in relation to the rail 86. The surface of the rail 86 includes first and second dimples 104, 106 that engage the ball 102 at different locations along the rail 86. The locations of the dimples 104, 106 are selected so that, when the ball 102 engages one of the dimples 104, 106, the measurement region detector 62 is aligned with a respective measurement region (e.g., the test region 16 or the control region 18) on the test strip 50 and the baseline region detector 64 is aligned with a respective region of the test strip 50 outside of any measurement region.

In a typical implementation, the support 82 also includes a second detent (not shown) that engages corresponding features on the rail 84 (also not shown).

FIG. 7A shows the support 82 sliding on the rail 86 in the direction of arrow 108 before the ball 102 engages the first dimple 104. FIG. 7B shows the position of the support 82 after the ball 102 is aligned with the first dimple 104. In this measurement first position, the spring 110 urges the ball 102 into the first dimple 104. The force exerted by the spring 110 resists movement of the support 82 along the rail 86 until a force is applied along the direction of the rail 86 that is sufficient to overcome the force of spring 110. In the first measurement position, the measurement region detector 62 is positioned directly over the control region 18 and the baseline region detector 64 is positioned directly over a respective baseline region of the detection zone 15 adjacent the control region 18 but outside any measurement region. In the first measurement position, the measurement region detector 62 produces signals in the measurement data channel in response to light received from the control region 18 and the baseline region detector 64 produces signals in the baseline data channel in response to light received from the adjacent baseline region. The measurement data channel signals and the baseline data channel signals may be produced contemporaneously or sequentially and are stored in the memory 47 of the diagnostic test system 40.

Upon application of a force in the direction 108 that is sufficient to overcome the force of the spring 110, the support 82 slides along the rail 86 in the direction 108 until it reaches a second measurement position where the spring 110 urges the ball 102 into the second dimple 106. In this second measurement position, the measurement region detector 62 is positioned directly over the test region 16 and the baseline region detector 64 is positioned directly over a respective baseline region of the detection zone 15 adjacent the test region 16 but outside any measurement region. In the second measurement position, the measurement region detector 62 produces signals in the measurement data channel in response to light received from the test region 16 and the baseline region detector 64 produces signals in the baseline data channel in response to light received from the adjacent baseline region. The measurement data channel signals and the baseline data channel signals may be produced contemporaneously or sequentially and are stored in the memory 47 of the diagnostic test system 40.

In some implementations of the embodiment shown in FIGS. 7A-7C, the support 82 is moved manually by a user of the diagnostic test system 40. When the spring-loaded ball 102 is aligned with the dimples 104, 106, the user will receive tactile feedback in the form of resistance to movement of the support 82 as a result of the engagement of the ball 102 with the dimples 104, 106. The user also may receive tactile feedback (e.g., vibrations) and audible feedback (e.g., clicks) that are produced by the engagement of the ball 102 with the surfaces of the dimples 104, 106. Such feedback will inform the user that the test strip 50 is in a measurement position with respect to the detection system 60 and that the detection system 60 may be activated so that measurements from the corresponding measurement and baseline regions may be obtained. After the measurements have been obtained, the user may apply sufficient force to overcome the force of the spring 110 and slide the support into the next measurement position.

In other implementations of the embodiment shown in FIGS. 7A-7C, the support 82 is moved automatically by a motor that is incorporated in the diagnostic test system 40. When the ball 102 is aligned with the dimples 104, 106, the spring 110 urges the ball 102 into the dimples 104, 106. The detection system 60 may be configured to detect when the ball 102 has engaged the dimples 104, 106. For example, in some embodiments, the extension of the spring 110 may actuate a piezoelectric element that sends an alignment signal to the detection system 60. Alternatively, the rail 86 may include electrical contacts in the dimples 104, 106 and when the ball 102 engages one of the electrical contacts the ball 102 may create a closed electrical circuit that is detected by the detection system 60. In response to the detection of the engagement of the ball 102 with the dimples 104, 106, the detection system 60 transmits a control signal that causes the motor to stop sliding the support 82 over the rails 84, 86. The detection system 60 then obtains measurements from the corresponding measurement and baseline regions. After the measurements have been obtained, the detection system 60 sends a control signal that reactivates the motor, which applies sufficient force to overcome the force of the spring 110 and slides the support 82 into the next measurement position.

Other alignment system embodiments correspond to the embodiment shown in FIGS. 7A-7C, except that each of these other embodiments includes only a respective one of the detent and the set of one or more dimples 104, 106.

2. Optical Position Markers

The optical position markers that are described below directly indicate the positions of the measurement regions on the test strip 50. With respect to these embodiments, the detection system 60 is configured to detect the optical position markers. In response to detection of the optical position markers, the detection system 60 triggers a perceptible (e.g., visible or audible) indication that the test strip 50 is in a measurement position. In other implementations, the detection system 60 deactivates a motor to stop the sliding of the support 82 over the rails 84, 86. The detection system 60 then obtains measurements from the corresponding measurement and baseline regions. After the measurements have been obtained, the user is notified or the motor is reactivated so that sufficient force may be applied to the support 82 to overcome the force of the spring 110 and slide the support 82 into the next measurement position.

FIG. 8 shows an implementation of the test strip 50 that includes an exemplary set of optical position markers 112, 114, 116, 118 that are positioned adjacent to the test region 16 and the control region 18. In particular, the optical position marker 112 is positioned adjacent to an upstream edge 120 of the test region 16 and the optical position marker 114 is positioned adjacent to a downstream edge 122 of the test region 16. Similarly, the optical position marker 116 is positioned adjacent to an upstream edge 124 of the control region 18 and the optical position marker 118 is positioned adjacent to a downstream edge 126 of the control region 18. In the illustrated embodiment, the optical position markers 112-118 are beside one edge of the detection zone 15. In other embodiments, the optical position markers 112-118 may be located in different regions of the detection zone 15.

In some implementations, the optical position markers 112-118 may have a detectable optical response that is different from the optical response of adjacent surface regions. For example, the optical position markers 112-118 may have a greater reflection or emission than adjacent surface regions with respect to light within a specified wavelength range (e.g., the visible wavelength range: 390 nm to 770 nm). In some implementations of this type, one or more of the optical positions markers 112-118 are implemented by retroreflectors. In other implementations, the optical position markers 112-118 may have a lower reflection or emission than adjacent surface regions with respect to light within the specified wavelength range. In some implementations, the optical position markers 112-118 are capable of fluorescent emission within a first wavelength range, whereas the adjacent surface regions are capable of fluorescent emission within a second wavelength range that is different from the first wavelength range or with an intensity that is significantly lower than the intensity of the fluorescent emission by the optical position markers within the first wavelength range. In some implementations, the optical position markers 112-118 are composed of quantum dots that exhibit fluorescent emission within narrow wavelength ranges. The optical position markers 112-118 may be formed on the exposed surface of the test strip 50 in any of a wide variety of different ways, including silk screening and other printing or deposition methods.

In the illustrated embodiment, the optical position markers 112-118 have square shapes. In general, however, the optical position markers 112-118 may have any type of shape, including a polygonal (e.g., rectangular) shape and a curved (e.g., elliptical or circular) shape. The shapes of the optical position markers 112-118 may be the same or different.

In some implementations, the data analyzer 46 is operable to identify the optical position markers 112-118 based on the sizes, shapes, and/or locations of the optical position markers 112-118. For example, the data analyzer 46 may identify the optical position markers 112-118 by locating square regions in an image of the detection zone 15 that is captured by one of the measurement region detector 62 and the baseline region detector 64 (or another optical detector in the is detection system 60). In other implementations, the data analyzer 46 may identify the optical position markers 112-118 based on one or more attributes (e.g., relative intensity, wavelength, or decay profile) of the light reflected or fluorescing from the optical position markers 112-118.

The data analyzer 46 readily may determine the bounds of the measurement regions 16, 18 based on the edges of the identified optical position markers 112-118 in an image that is captured by one of the measurement region detector 62 and the baseline region detector 64 (or another optical detector in the detection system 60). For example, with respect to the implementation illustrated in FIG. 8, the data analyzer 46 identifies the test region 16 as the transverse region between the optical position markers 112, 114 and identifies the control region 18 as the transverse region between the optical position markers 116, 118.

In some embodiments, only the upstream optical position markers 112, 116 are present. With respect to these embodiments, the data analyzer 46 identifies the test and control 16, 18 as corresponding to the transverse regions immediately following the optical position markers 112, 116 with respect to the lateral flow direction. In other embodiments, only the downstream optical position markers 114, 118 are present. With respect to these embodiments, the data analyzer 46 identifies the test and control 16, 18 as corresponding to the transverse regions immediately preceding the optical position markers 114, 118 with respect to the lateral flow direction 51.

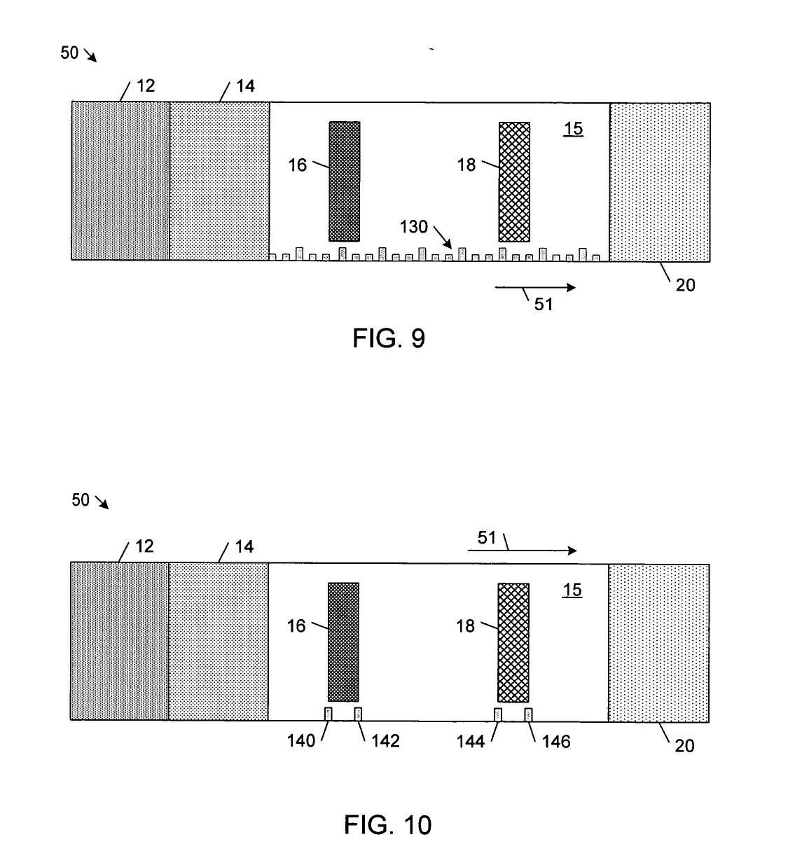

FIG. 9 shows an implementation of the test strip 50 that includes an exemplary set of optical position markers 130 that are spaced regularly along an edge of the test strip 50 along the lateral flow direction. The optical position markers 130 include features that have a different reflection or emission characteristic than the surface of the test strip 50. As a result, the measurements that are obtained near the edge of the test strip 50 vary in intensity in accordance with the pattern of the optical position markers 130. In this way, the optical position markers 130 encode positions along the test strip 50 in the lateral flow direction 51. With respect to the implementation shown in FIG. 9, the data analyzer 46 may determine the encoded positions along the lateral flow direction by incrementing a position counter with each intensity variation cycle (e.g., peak-to-valley) in the light intensity measurements obtained from the edge of the detection zone 15.

In these implementations, the data analyzer 46 correlates the light intensity measurements with the positions along the test strip 50 in the lateral flow direction 51. The location correlation information may be stored in a lookup table that is indexed by the position counter value. Based on this information and on the predetermined information correlating the locations of the measurement regions with the light intensity contrast pattern produced by the optical position markers 130, the data analyzer 46 can identify the corresponding measurement regions.

In other implementations, the optical position markers 130 may encode position information in variations in the lengths of the optical position markers along the lateral flow direction 51. Alternatively, the optical position markers 130 may encode position information in variations in the spacing between adjacent ones of the optical position markers 130 along the lateral flow direction 51.

3. Electrical Position Markers

In some embodiments, the test strip 50 includes electrical position markers that are aligned with respective measurement regions on the test strip 50 and thereby directly indicate the positions of the measurement regions on the test strip 50. With respect to these embodiments, the detection system 60 is configured to detect the electrical position markers. In response to detection of the electrical position markers, the detection system 60 triggers a perceptible (e.g., visible or audible) indication that the test strip 50 is in a measurement position. In other implementations, the detection system 60 deactivates a motor to stop the sliding of the support 82 over the rails 84, 86. The detection system 60 then obtains measurements from the corresponding measurement and baseline regions. After the measurements have been obtained, the user is notified or the motor is reactivated so that sufficient force may be applied to the support to overcome the force of the spring 110 and slide the support 82 into the next measurement position.

FIG. 10 shows an implementation of the test strip 50 that includes an exemplary set of electrical position markers 140, 142, 144, 146 that are spaced along the edge of the test strip 50. The electrical position markers 140-146 include features that have a different electrical characteristic than the adjacent areas on the surface of the test strip 50. As a result, the measurements that are obtained near the edge of the test strip 50 vary in electrical response in accordance with the pattern of the electrical position markers 140-146. With respect to these embodiments, the detection system 60 includes an electrical detection system that is capable of detecting the electrical position markers. In general, any type of electrical conductor detection method may be used to detect the electrical position markers, including current, voltage, resistance, and capacitance based measurement methods.

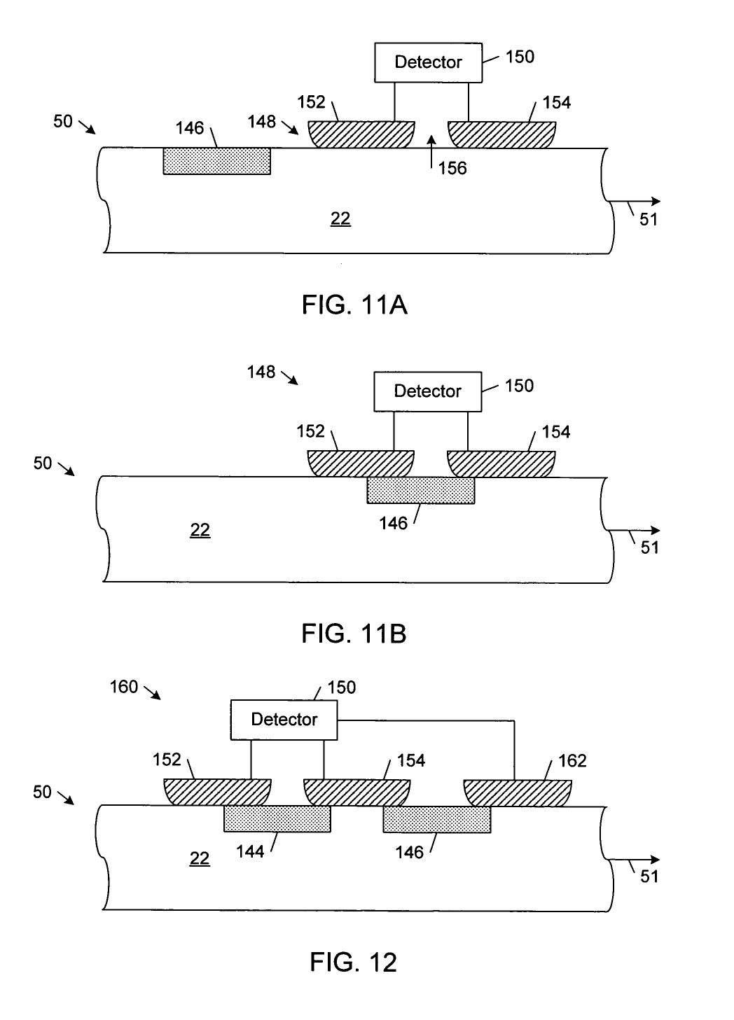

FIG. 11A shows an embodiment of an electrical detection system 148 disposed on a portion of an embodiment of the test strip shown in FIG. 11A. The electrical detection system 148 includes a detector 150, a first electrical contact 152, and a second electrical contact 154. The first and second electrical contacts 152, 154 are electrically connected to the detector 150 and are separated from one another by an air gap 156, which forms an open circuit. The detector 150 may include any type of circuit (e.g., an ohmmeter, a voltmeter, and an ammeter) that is capable of detecting when an electrical connection is formed across the air gap 156. In these embodiments, the top surface of the test strip is formed of a material with a high electrical resistance except at the locations of the electrical position markers 140-146.

In operation, at least one of the electrical detection system 148 and the test strip 50 is moved relative to the other in a direction parallel to the lateral flow direction 51. The first and second electrical contacts 152, 154 slide over the top surface of the test strip. In some implementations, the first and second electrical contacts 152, 154 are urged (e.g., by springs) against the top surface of the test strip. In the position shown in FIG. 11A, the first and second electrical contacts 152, 154 are connected only by the material of the top surface of the test strip. In this position, the detector 150 is configured to determine that there is an open circuit between the first and second electrical contacts 152, 154. In the position shown in FIG. 11B, on the other hand, the first and second electrical contacts 152, 154 are connected by the electrical position marker 146. In this position, the detector 150 is configured to determine that there is a closed circuit between the first and second electrical contacts 152, 154.

The detector 150 may determine whether there is an open circuit or a closed circuit between the first and second electrical contacts 152, 154 by comparing an electrical measurement (e.g., current, voltage, or resistance) between the first and second electrical contacts 152, 164 to a threshold value. For example, the detector may determine that there is an open circuit between the first and second electrical contacts 152, 154 when the measured electrical resistance value is greater than or equal to a threshold resistance value and that there is a closed circuit between the first and second electrical contacts 152, 154 when the measured electrical resistance value is below the threshold value.

In the embodiment shown in FIG. 10, the electrical position markers 140-146 are aligned with the upstream and downstream edges of the test region 16 and the control region 18 along the lateral flow direction 51. In this way, the data analyzer 46 readily may determine that the test and control regions 16, 18 are located between the detected positions of the electrical position markers 140, 142 and 144, 146, respectively.

FIG. 12 shows an embodiment of an electrical detection system 160 that corresponds to the electrical detection system 148, except that the electrical detection system 160 includes a third electrical contact 162. In this embodiment, the detector 150 determines that the test strip 50 is in a measurement position only when there is a closed circuit between the first, second, and third electrical contacts 152, 154, 162. That is when the first, second, and third electrical contacts 152, 154, 162 are electrically connected by a pair of electrical position markers (e.g., the position markers 144, 146), as shown in FIG. 12. When the electrical detection system 160 detects an open circuit between one or more pairs of the first, second, and third electrical contacts 152, 154, 162, the electrical detection system 160 determines that the test strip 50 is not in a measurement position.

In other embodiments, the electrical position markers may encode position information in different ways. For example, in some embodiments, the electrical position markers may be positioned at regularly spaced locations along the edge of the test strip 50. As a result, the electrical measurements that are obtained near the edge of the test strip 50 vary in value in accordance with the pattern of the electrical position markers. In this way, the electrical position markers encode positions along the test strip 50 in the lateral flow direction 51. With respect to these embodiments, the data analyzer 46 may determine the encoded positions along the lateral flow direction 51 by incrementing a position counter with each measurement variation cycle (e.g., peak-to-valley) in the electrical measurements obtained from the edge of the detection zone 15.

IV. Conclusion

The embodiments that are described in detail above are capable of respectively obtaining measurement signals and baseline signals from measurement and baseline regions of an assay test strip without requiring complex and resource intensive analysis of the signal data.

Other embodiments are within the scope of the claims.

1. A diagnostic test system for assaying a test strip having a flow path for a fluid sample, a sample receiving zone coupled to the flow path, and a detection zone coupled to the flow path and having at least one measurement region, the diagnostic test system comprising:
a detection system comprising a measurement region detector and a baseline region detector each operable to produce signals in response to light from the test strip;
an alignment system configured cooperatively with the detection system to hold the test strip in a respective measurement position relative to the detection system for each measurement region in the detection zone, wherein in each measurement position the measurement region detector produces measurement signals in a measurement data channel in response to light from a respective measurement region of the test strip and the baseline region detector produces baseline signals in a baseline data channel separate from the measurement data channel in response to light from a respective region of the test strip outside of any measurement region; and
a data analyzer operable to quantify respective ones of the measurement signals with respect to corresponding ones of the baseline signals for each measurement position of the test strip relative to the detection system.

2. The diagnostic test system of claim 1, wherein in each measurement position the baseline region detector receives light from a respective region in the detection zone adjacent to the measurement region from which the measurement region detector receives light.

3. The diagnostic test system of claim 1, wherein in each measurement position the measurement region detector is positioned directly over a respective measurement region in the detection zone and the baseline region detector is positioned directly over a respective region in the detection zone outside of any measurement region.

4. The diagnostic test system of claim 1, wherein in each measurement position the measurement region detector and the baseline region detector simultaneously produce the measurement signals and the baseline signals.

5. The diagnostic test system of claim 1, wherein the alignment system is configured cooperatively with the detection system to hold the test strip in multiple measurement positions relative to the detection system, wherein in each of the multiple measurement positions the measurement region detector receives light from a respective measurement region in the detection zone and the baseline region detector receives light from a respective region of the test strip outside of any measurement region.

6. The diagnostic test system of claim 1, wherein the alignment system comprises a support configured to hold the test strip.

7. The diagnostic test system of claim 6, wherein the support is configured to move relative to the detection system.

8. The diagnostic test system of claim 7, wherein the alignment system comprises at least one rail, and the support is configured to slide on the at least one rail.

9. The diagnostic test system of claim 8, wherein the alignment system comprises a detent operable to resist movement of the support when the support holds the test strip in a respective measurement position.

10. The diagnostic test system of claim 1, wherein the detection system is operable to detect alignment features indicating locations of respective ones of the measurement regions on the test strip.

11. The diagnostic test system of claim 10, wherein the detection system is operable to optically detect the alignment features.

12. The diagnostic test system of claim 10, wherein the detection system is operable to electrically detect electrically conducting alignment features on the test strip.

13. The diagnostic test system of claim 12, wherein the detection system comprises at least two electrical contacts configured to contact one or more of the electrically conducting alignment features when the test strip is in a measurement position.

14. The diagnostic test system of claim 10, further comprising a motor that provides relative movement between the detection system and the alignment system, wherein the detection system is operable to trigger deactivation of the motor in response to detection of ones of the alignment features.

15. The diagnostic test system of claim 10, wherein the detection system is operable to trigger a perceptible alarm alerting a user that the test strip is in a respective measurement position in response to detection of ones of the alignment features.

16. The diagnostic test system of claim 1, wherein the data analyzer is operable to compare a measurement signal produced by the measurement region detector in response to light from a test region of the test strip to a baseline signal.

17. The diagnostic test system of claim 1, wherein the data analyzer is operable to compare a measurement signal produced by the measurement region detector in response to light from a test region of the test strip to a measurement signal produced by the measurement region detector in response to light from a control region of the test strip.

18. A diagnostic test method for assaying a test strip having a flow path for a fluid sample, a sample receiving zone coupled to the flow path, and a detection zone coupled to the flow path and having at least one measurement region, the diagnostic test method comprising:
for each measurement region in the detection zone, holding the test strip in a respective measurement position;
in each measurement position, producing measurement signals in a measurement data channel in response to light from a respective measurement region of the test strip and producing baseline signals in a baseline data channel separate from the measurement data channel in response to light from a respective region of the test strip outside of any measurement region; and
quantifying respective ones of the measurement signals with respect to corresponding ones of the baseline signals for each measurement position.

19. The method of claim 18, further comprising moving the test strip into each of multiple measurement positions.

20. The method of claim 19, further comprising in each of the measurement positions resisting movement of the test strip.

21. The method of claim 18, further comprising detecting alignment features indicating locations of respective ones of the measurement regions.

22. The method of claim 21, further comprising terminating movement of the test strip at each of the measurement positions in response to detection of the respective one of the alignment features.

23. The method of claim 21, further comprising triggering a perceptible alarm alerting a user that the test strip is in a respective measurement position in response to detection of ones of the alignment features.

24. The method of claim 18, wherein the quantifying comprises comparing respective ones of the measurement signals produced in response to light from a test region of the test strip to corresponding ones of the measurement signals produced in response to light from a control region of the test strip.
